(19) United States
(12) Patent Application Publication
(10) Pub. No.: US 2021/0169383 A1
(43) Pub. Date: Jun. 10, 2021

(54) BLOOD GLUCOSE MEASUREMENT DEVICE

(71) Applicant: Samsung Electronics Co., Ltd., Suwon-si, Gyeonggi-do (KR)

(72) Inventors: Seong Je CHO, Suwon-si (KR); Byeong Hoon KWAK, Suwon-si (KR); Young Jae OH, Suwon-si (KR); Chul Ho CHO, Suwon-si (KR); Hyoung Seon CHOI, Suwon-si (KR)

(21) Appl. No.: 16/770,761

(22) PCT Filed: Oct. 26, 2018
(86) PCT No.: PCT/KR2018/012823

§ 371 Date: Jun. 8, 2020

(30) Foreign Application Priority Data
Jan. 8, 2018 (KR) 10-2018-0002224

(51) Int. Cl. A61B 5/145; A61B 5/0507

(52) U.S. Cl. CPC A61B 5/14532; A61B 5/7203; A61B 5/0507

(57) ABSTRACT

A blood glucose measurement device is disclosed. The present blood glucose measurement device comprises: a substrate; a first resonance sensor and a second resonance sensor arranged on the substrate; and a shield layer arranged below the second resonance sensor with respect to the direction from the second resonance sensor toward a subject.

### TECHNICAL FIELD

The disclosure relates to a blood glucose measurement device, and more particularly to a non-invasive blood glucose measurement device capable of more accurately measuring blood glucose by removing a noise of an external environment.

### BACKGROUND ART

The quantitative determination of an analyte in biological fluids is useful for diagnosis and treatment of physiologic abnormality. For example, an amount of glucose (blood glucose) should be periodically checked for diagnosis and prevention of diabetes.

As a blood glucose measurement device, a device measuring blood glucose by drawing blood is provided. In the type requiring the blood drawing, there were problems that a measured value of blood glucose may vary depending on proficiency in drawing blood and it is not possible to perfectly detect a change in concentration of a measurement target material in the blood only by several times of intermittent measurement.

Accordingly, a blood glucose measurement device capable of monitoring a concentration of a measurement target material accurately without drawing blood has been developed, and the blood glucose measurement device typically includes a fully implantable type for fully implanting a blood glucose measurement device into the body and a minimally invasive type for inserting a needle-like sensor insertable to the subcutaneous tissue.

However, such invasive types had problems that there is a risk of entering of external materials and a pain may come due to a needle.

### DISCLOSURE

### Technical Problem

The disclosure has been made to solve the aforementioned problems, and an object of the disclosure is to provide a non-invasive blood glucose measurement device capable of more accurately measuring blood glucose by removing a noise of an external environment.

### Technical Solution

According to an embodiment of the disclosure for achieving the aforementioned object, there is provided a blood glucose measurement device including a substrate, a first resonance sensor and a second resonance sensor arranged on the substrate; and a shield layer arranged below the second resonance sensor with respect to a direction from the second resonance sensor toward a subject.

The first resonance sensor may be arranged next to the second resonance sensor in parallel.

The first resonance sensor may be arranged below the shield layer with respect to the direction from the second resonance sensor toward a subject.

The blood glucose measurement device according to the embodiment may further include an electromagnetic wave transmission layer arranged below the first resonance sensor with respect to a direction from the first resonance sensor toward a subject.

The shield layer and the electromagnetic wave transmission layer may be formed at the same height.

The shield layer may be formed to be embedded in the substrate.

The shield layer may be formed of a ferrite.

The blood glucose measurement device according to the embodiment may further include a processor configured to, based on a subject reaching or coming into contact with the first resonance sensor and the second resonance sensor, obtain information regarding a blood glucose level of the subject based on a shift of a resonance frequency of each of the first resonance sensor and the second resonance sensor.

The processor may be configured to, based on a subject reaching or coming into contact with the first resonance sensor and the second resonance sensor, subtract a shift value of the resonance frequency of the second resonance sensor from a shift value of the resonance frequency of the first resonance sensor, and obtain information regarding a blood glucose level of the subject corresponding to the subtracted value.

The blood glucose measurement device according to the embodiment may further include a communicator comprising circuitry; and a processor configured to, based on a subject reaching or coming into contact with the first resonance sensor and the second resonance sensor, control the communicator to transmit a shift of a resonance frequency of each of the first resonance sensor and the second resonance sensor to an external device.

The blood glucose measurement device according to the embodiment may further include a strain sensor for detecting a shape strain of the first resonance sensor and the second resonance sensor.

The strain sensor may comprise a first strain sensor arranged to be adjacent to the first resonance sensor and a second strain sensor arranged to be adjacent to the second resonance sensor.

The first strain sensor may be arranged to surround the first resonance sensor and the second strain sensor is arranged to surround the second resonance sensor.

The blood glucose measurement device according to the embodiment may further include a processor configured to, based on a subject reaching or coming into contact with the first resonance sensor and the second resonance sensor, obtain information regarding a blood glucose level of the subject based on a shift of a resonance frequency of each of the first resonance sensor and the second resonance sensor and sensed data obtained through the strain sensor.

The processor may be configured to obtain a shift value of a resonance frequency due to shape strain of each of the first resonance sensor and the second resonance sensor based on sensed data obtained by the strain sensor, and, based on a subject reaching or coming into contact with the first resonance sensor and the second resonance sensor, obtain information regarding a blood glucose level of the subject by subtracting a shift value of the resonance frequency due to the shape strain from a shift value of the resonance frequency of each of the first resonance sensor and the second resonance sensor.

### DESCRIPTION OF DRAWINGS

FIG. 1 is a view for describing a blood glucose measurement device according to an embodiment,

FIGS. 2 to 4 are views showing cross sectional views of the blood glucose measurement device according to various embodiments,

FIG. 5 is a view for describing the blood glucose measurement device according another embodiment,

FIG. 6 is a view showing a cross sectional view of the blood glucose measurement device described in FIG. 5,

FIG. 7 is a view for describing the blood glucose measurement device according to an embodiment additionally including a strain sensor,

FIG. 8 is a view for describing the blood glucose measurement device according to another embodiment additionally including a strain sensor,

FIG. 9 is a view showing a cross sectional view of the blood glucose measurement device described in FIG. 8,

FIG. 10 is a block diagram for describing a configuration of the blood glucose measurement device according to an embodiment,

FIG. 11 is a view for describing a sensor unit of the blood glucose measurement device according to an embodiment,

FIGS. 12 and 13 are views for describing a shift in resonance frequency of a first resonance sensor and a second resonance sensor according to an embodiment, and

FIG. 14 is a view for describing an external device connected to the blood glucose measurement device according to an embodiment.

### DETAILED DESCRIPTION OF EXEMPLARY EMBODIMENTS

Hereinafter, embodiments of the disclosure will be described with reference to the accompanying drawings. It should be noted that the technologies disclosed in this disclosure are not for limiting the scope of the disclosure to a specific embodiment, but they should be interpreted to include all modifications, equivalents and/or alternatives of the embodiments of the disclosure. In relation to explanation of the drawings, similar reference numerals may be used for similar elements.

In this disclosure, the terms such as “comprise”, “may comprise”, “consist of”, “or may consist of” are used herein to designate a presence of corresponding features (e.g., constituent elements such as number, function, operation, or part), and not to preclude a presence of additional features.

In this disclosure, expressions such as “A or B”, “at least one of A [and/or] B,”, or “one or more of A [and/or] B,” include all possible combinations of the listed items. For example, “A or B”, “at least one of A and B,”, or “at least one of A or B” includes any of (1) at least one A, (2) at least one B, or (3) at least one A and at least one B.

The expressions “first,” “second” and the like used in the disclosure may denote various elements, regardless of order and/or importance, and may be used to distinguish one element from another, and does not limit the elements. For example, a first user device and a second user device may indicate different user devices, regardless of order and/or importance. For example, a first element may be referred to as a second element and the second element may also be similarly referred to as the first element, while not departing from the scope of a right of the disclosure.

If it is described that a certain element (e.g., first element) is “operatively or communicatively coupled with/to” or is “connected to” another element (e.g., second element), it should be understood that the certain element may be connected to the other element directly or through still another element (e.g., third element). In contrast, if it is described that a certain element (e.g., first element) is “directly coupled to” or “directly connected to” another element (e.g., second element), it may be understood that there is no element (e.g., third element) between the certain element and the another element.

Also, the expression “configured to” used in the disclosure may be interchangeably used with other expressions, for example, “suitable for,” “having the capacity to,” “designed to,” “adapted to,” “made to,” and “capable of,” depending on cases. Meanwhile, the expression “configured to” does not necessarily mean that a device is “specifically designed to” in terms of hardware. Instead, under some circumstances, the expression “a device configured to” may mean that the device “is capable of” performing an operation together with another device or component. For example, the phrase “a processor configured (or set) to perform A, B, and C” may mean a dedicated processor (e.g., an embedded processor) for performing the corresponding operations, or a generic-purpose processor (e.g., a central processing unit (CPU) or an application processor) that can perform the corresponding operations by executing one or more software programs stored in a memory device.

A term such as “module”, “unit”, or “part” in the disclosure may be used to refer to an element performing at least one function or operation, and may be implemented as hardware, software, or a combination of hardware and software. Further, except for when each of a plurality of “modules”, “units”, “parts” and the like needs to be realized in individual hardware, the components may be integrated in at least one module or chip and be implemented in at least one processor.

The terms used in the disclosure are used to merely describe a specific embodiment and may not be used to limit the scope of other embodiments. Unless otherwise defined specifically, a singular expression may encompass a plural expression. The terms used herein including technical or scientific terms may have the same meaning as those normally understood by those skilled in the art in the technical field disclosed herein. Among the terms used herein, the terms defined in general dictionaries may be interpreted as the same or similar meaning as the contextual meaning in the related art, and may not be interpreted as ideal or extremely formal meaning, unless clearly defined in the disclosure. Depending on cases, even if it is a term defined in the disclosure, it may not be interpreted to preclude embodiments of the disclosure.

Hereinafter, a blood glucose measurement device according to an embodiment of the disclosure will be described.

The blood glucose measurement device according to an embodiment of the disclosure may be implemented in various forms of electronic devices.

The blood glucose measurement device may be implemented, for example, a smart phone, a PC tablet personal computer, a mobile phone, a video phone, an e-book reader, a desktop personal computer, a laptop personal computer (PC), a netbook computer, a workstation, a personal digital assistant (PDA), a portable multimedia player (PMP), an MP3 player, a mobile medical device, a camera, or a wearable device. According to various embodiments, the wearable device may include at least one of an accessory type (e.g., a watch, a ring, a bracelet, an ankle bracelet, a necklace, a pair of glasses, a contact lens or a head-mounted-device (HMD)), a fabric or a garment-embedded type (e.g., electronic cloth), a skin-attached type (e.g., a skin pad or a tattoo), or a bio-implant type (implantable circuit).

In some embodiments, the blood glucose measurement device may be a home appliance. The home appliance may include at least one of, for example, a television, a digital video disk (DVD) player, an audio system, a refrigerator, air-conditioner, a vacuum cleaner, an oven, a microwave, a washing machine, an air purifier, a set top box, a home automation control panel, a security control panel, a TV box (e.g., SAMSUNG HomeSync™, APPLE TV™, or GOOGLE TV™), a game console (e.g., XBOX™, PLAYSTATION™), an electronic dictionary, an electronic key, a camcorder, or an electronic frame.

The blood glucose measurement device may be one or a combination of various devices described above. The blood glucose measurement device according to an embodiment may be a flexible electronic device. In addition, the blood glucose measurement device according to the embodiment of the disclosure is not limited to the devices described above and may include a new electronic device along technology development.

The blood glucose measurement device of the disclosure use a method for measuring blood glucose by a non-invasive method without a directly contact with blood, in order to solve a problem of a blood drawing method or an invasive method described in the section of the background art.

For such non-invasive blood glucose measurement, the blood glucose measurement device of the disclosure measures a blood glucose level of a subject by using electromagnetism based on a property of a subject (that may be a specific part of a body for measuring blood glucose, for example, a finger. In addition, various body tissue such as skin/fat/muscle may be a subject) changing in dielectric property according to a change of blood glucose. Specifically, the blood glucose measurement device of the disclosure includes a resonator consisting of a resonance sensor. When a subject reaches or comes into contact with a resonance sensor, the resonance sensor and the subject configure an inductively coupled resonance circuit, and accordingly, a resonance frequency of the resonance sensor shifts. Such a shift of the resonance frequency varies depending on the dielectric property of the subject, and the blood glucose level of the subject may be measured by observing a shift of a dielectric frequency of the subject.

The resonance frequency of the resonance sensor may shift due to other factors. For example, the resonance frequency of the resonance sensor may shift due to external environmental factors such as a temperature change or humidity change around the resonance sensor, and the resonance frequency may also shift according to physical shape strain of the resonance sensor due to an external force.

An object of the disclosure is to measure an accurate blood glucose level of a subject by removing a shift in resonance frequency due to factors other than the blood glucose level of the subject described above.

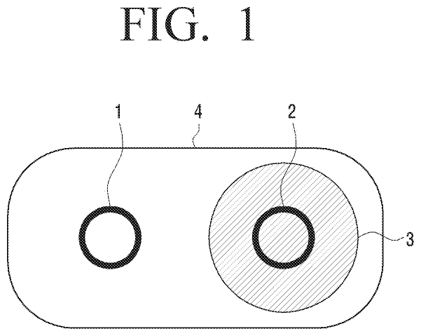

FIG. 1 is a view for describing a structure of a blood glucose measurement device according to an embodiment of the disclosure.

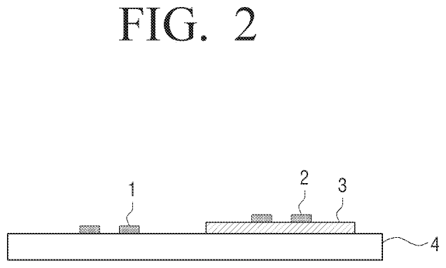

Referring to FIG. 1, a blood glucose measurement device includes a substrate 4, a first resonance sensor 1 and a second resonance sensor 2 arranged on the substrate 4. The blood glucose measurement device also includes a shield layer 3 arranged below the second resonance sensor 2 with respect to a direction from the second resonance sensor 2 toward the subject. FIG. 2 is a cross sectional view of the structure of FIG. 1.

The first resonance sensor 1 and the second resonance sensor 2 are distinguished with the expressions “first” and “second”, but the first resonance sensor 1 and the second resonance sensor 2 may be the same sensor.

The shield layer 3 is arranged below the second resonance sensor 2, whereas the shield layer is not arranged below the first resonance sensor 1.

The shield layer 3 is an element for shielding electromagnetic waves. A material constituting the shield layer 3 may be any material as long as it is metal or ceramic dielectric substance capable of reflecting or absorbing electromagnetic waves. For example, the shield layer 3 may be formed of a material such as a ferrite, sendust, or a nickel alloy.

A thickness of the shield layer 3 may be determined by considering a skin effect or a penetration depth or a skin depth. The skin effect is a phenomenon in that electromagnetic wave is unable to enter inside and remains in the vicinity of the surface, as the frequency of the electromagnetic wave increases. The penetration depth is a depth by which the electromagnetic waves averagely penetrate.

The penetration depth δ may be obtained from a mathematical equation 1 as shown below.

$$δ = \sqrt{\frac{2}{ωμσ}} \qquad Mathematical equation 1$$

In the mathematical equation 1, μ is a permeability and σ is an electrical conductivity.

The table 1 below shows penetration depths according to frequencies of exemplified metal substances used as the material of the shield layer 3.

TABLE 1

| | | Frequency (GHz) | | | | | | | |
|---|---|---|---|---|---|---|---|---|---|
| | | 0.1 | 0.3 | 0.5. | 1 | 3 | 5 | 10 | 30 |
| Penetration | Silver | 6.44 | 3.72 | 2.88 | 2.04 | 1.18 | 0.91 | 0.64 | 0.37 |
| depth (μm) | Copper | 6.61 | 3.82 | 2.96 | 2.09 | 1.21 | 0.93 | 0.66 | 0.38 |
| | Gold | 7.86 | 4.54 | 3.52 | 2.49 | 1.44 | 1.11 | 0.79 | 0.45 |
| | Aluminum | 7.96 | 4.59 | 3.56 | 2.52 | 1.45 | 1.13 | 0.80 | 0.46 |
| | Brass | 9.87 | 5.70 | 4.41 | 3.12 | 1.80 | 1.40 | 0.99 | 0.57 |
| | Nickel | 13.00 | 7.50 | 5.81 | 4.11 | 2.37 | 1.84 | 1.30 | 0.75 |
| | Iron | 15.92 | 9.19 | 7.12 | 5.03 | 2.91 | 2.25 | 1.59 | 0.92 |
| | Platinum | 16.59 | 9.58 | 7.42 | 5.25 | 3.03 | 2.35 | 1.66 | 0.96 |
| | Tin | 16.78 | 9.69 | 7.50 | 5.31 | 3.06 | 2.37 | 1.68 | 0.97 |
| | Lead | 22.51 | 13.00 | 10.07 | 7.12 | 4.11 | 3.18 | 2.25 | 1.30 |

In the implementation, the shield layer 3 may be formed to have a thickness that is three or four times the penetration depth for more complete shielding. The sufficient shielding effect may be exhibited when the shield layer 3 is formed with a thickness of several tens μm. The thickness of the shield layer 3 is preferably thin, since the entire size of the blood glucose measurement device may be minimized.

The second resonance sensor 2 does not show a shift in resonance frequency according to an effect of a subject by the shield layer 3. Instead, the second resonance sensor 2 may be exposed to the external environment such as a temperature or humidity in the same manner as the first resonance sensor 1, and show a shift in resonance frequency according thereto. Accordingly, the second resonance sensor 2 may be used for removing a noise due to the external environment in the shift in resonance frequency of the first resonance sensor 1 when measuring the blood glucose level of the subject. Therefore, it is possible to measure a more accurate blood glucose level.

The first resonance sensor 1 and the second resonance sensor 2 may consist of a ring-shaped resonator. For example, the ring-shaped resonator may be manufactured as a lumped element (LC) resonator since the resonator is formed of a copper wire coated with silver and has a cut part (gap).

The first resonance sensor 1 and the second resonance sensor 2 may have a ring shape as shown in FIG. 1, but are not limited thereto, and may be formed in various shapes.

The substrate 4 is for supporting the first resonance sensor 1 and the second resonance sensor 2. A surface opposite to the surface, where the first resonance sensor 1 and the second resonance sensor 2 are arranged, is a surface facing the subject. The substrate 4 may be formed of any material for supporting the first resonance sensor 1 and the second resonance sensor 2 among substances capable of transmitting the electromagnetic waves. The substrate 4 may be formed of a hard material or may be formed of a flexible material depending on the implementation.

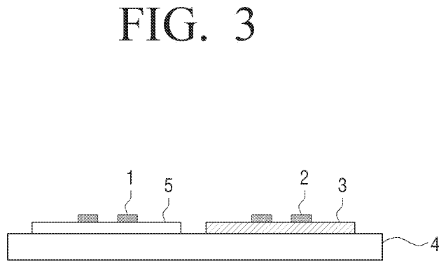

FIG. 3 is a view for describing a structure of the blood glucose measurement device according to another embodiment of the disclosure.

Compared to the embodiment described above with reference to FIG. 2, the embodiment described with reference to FIG. 3 further includes an electromagnetic wave transmission layer 5 arranged below the first resonance sensor 1 with respect to a direction from the first resonance sensor 1 toward a subject.

The electromagnetic wave transmission layer 5 may be manufactured with any material, as long as it is a material capable of transmitting the electromagnetic waves.

An object of the arrangement of the electromagnetic wave transmission layer 5 is to provide the second resonance sensor 2 and the first resonance sensor 1 in the same environment as possible. In a case of the embodiment described with reference to FIG. 2, the first resonance sensor 1 and the second resonance sensor 2 may be differently affected by a temperature conducted from the subject, since the first resonance sensor 1 and the second resonance sensor 2 are arranged at heights different from each other. As a result, a blood glucose level may be inaccurately measured. In contrast, according to the embodiment described with reference to FIG. 3, a blood glucose level may be more accurately measured, since the first resonance sensor 1 and the second resonance sensor 2 are arranged at the same height.

The shield layer 3 may be formed at the same height as that of the electromagnetic wave transmission layer 5. The same height herein does not mean only the exact same height, but also allows a difference in error range.

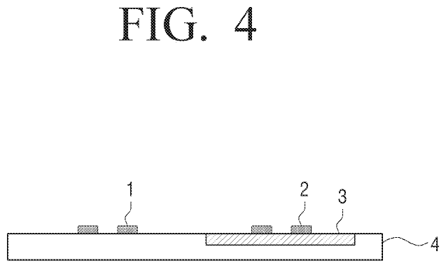

FIG. 4 is a view for describing a structure of the blood glucose measurement device according to still another embodiment of the disclosure.

Referring to FIG. 4, the shield layer 3 is embedded in the substrate 4 so that the first resonance sensor 1 and the second resonance sensor 2 are arranged at the same height. In the embodiment, the first resonance sensor 1 and the second resonance sensor 2 are arranged in the same environment, compared to the embodiment described with reference to FIG. 2, and it is advantageous that the entire height of the blood glucose measurement device may be reduced, compared to the embodiment described with reference to FIG. 3.

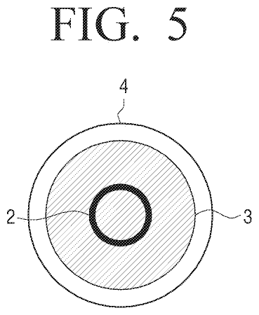

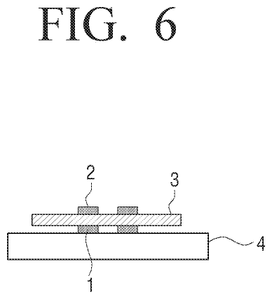

In the embodiments described above, the first resonance sensor 1 and the second resonance sensor 2 are arranged next to each other in parallel, but according to still another embodiment of the disclosure, the first resonance sensor 1 and the second resonance sensor 2 may be arranged vertically. This will be described with reference to FIGS. 5 and 6 below.

FIGS. 5 and 6 are views for describing a structure of the blood glucose measurement device according to an embodiment of the disclosure.

FIG. 5 is a top view and FIG. 6 is a cross sectional view of the structure of FIG. 5.

Referring to FIGS. 5 and 6, the first resonance sensor 1 may be arranged below the shield layer 3 with respect to a direction from the second resonance sensor 2 toward a subject. That is, the second resonance sensor 2 is arranged at the top, the shield layer 3 is arranged below this, and the first resonance sensor 1 is arranged below this. According to the embodiment, it is advantageous that the entire area of the blood glucose measurement device may be reduced.

The resonance frequency may also shift according to physical shape strain of the resonance sensor due to an external force, not only the surrounding environment factors such as a temperature or humidity. According to an embodiment of the disclosure, the blood glucose measurement device may include a strain sensor in order to compensate the shift due to the external force.

The strain sensor is a machine for showing a mechanical strain as an electric signal. When the strain sensor is attached to a surface of a structure, the strain sensor is able to measure a minimal change occurring on the surface thereof.

The strain sensor may be arranged to detect the shape strain of the first resonance sensor 1 and the second resonance sensor 2. According to an embodiment, a common strain sensor may be used for the first resonance sensor 1 and the second resonance sensor 2. According to still another embodiment, the strain sensor according to the disclosure may include a strain sensor for sensing the shape strain of the first resonance sensor 1 and a strain sensor for sensing the shape strain of the second resonance sensor 2, for more accurate measurement. In such a case, the strain sensor for sensing the shape strain of the first resonance sensor 1 may be arranged to be adjacent to the first resonance sensor 1, and the strain sensor for sensing the shape strain of the second resonance sensor 2 may be arranged to be adjacent to the second resonance sensor 2.

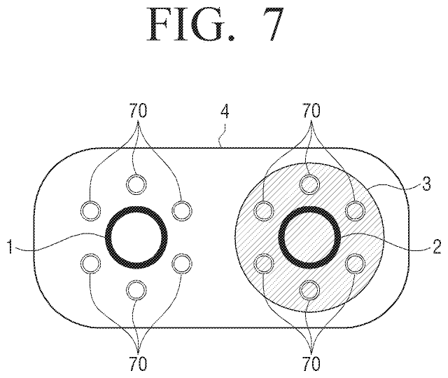

FIG. 7 is a view for describing arrangement of the strain sensors according to an embodiment of the disclosure. FIG. 7 is for describing additional arrangement of the strain sensors in the embodiment described with reference to FIG. 1.

Referring to FIG. 7, a plurality of strain sensors 70 may be arranged to surround the first resonance sensor 1, and a plurality of strain sensors 70 may be arranged to surround the second resonance sensor 2. The strain state of the first resonance sensor 1 may be determined based on a sensed value obtained by the plurality of strain sensors 70 arranged to surround the first resonance sensor 1, and the strain state of the second resonance sensor 2 may be determined based on a sensed value obtained by the plurality of strain sensors 70 arranged to surround the second resonance sensor 2. The strain sensors 70 for measuring the strain of the second resonance sensor 2 may be arranged on the shield layer 3. The shape of the strain sensor 70 is shown as a circle, but this is merely an embodiment, and there is no limitation thereto.

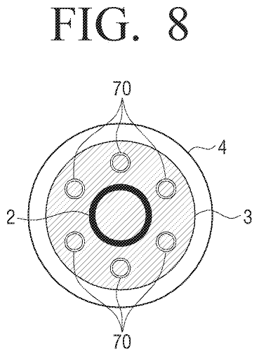

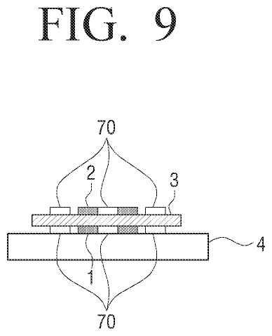

FIG. 8 is a view showing additional arrangement of strain sensors in the embodiment described with reference to FIG. 5, and FIG. 9 is a cross sectional view of the structure of FIG. 8. Referring to FIGS. 8 and 9, the strain sensors 70 are arranged to surround the first resonance sensor 1 and the second resonance sensor 2. The strain sensors 70 for measuring the strain of the second resonance sensor 2 may be arranged on the shield layer 3.

According to the embodiments described above, it is possible to remove a noise due to the surrounding environment such as a temperature or humidity and to remove a noise due to an external force.

In the above embodiments, it is described that the second resonance sensor 2 is included, but if it is not necessary to remove a noise due to the surrounding environment such as a temperature or humidity, but it is only necessary to remove a noise due to an external force, the second resonance sensor 2 and the shield layer 3 may be omitted, and the blood glucose measurement device may consist of the first resonance sensor 1 and the strain sensor 70 for detecting the shape strain of the first resonance sensor 1.

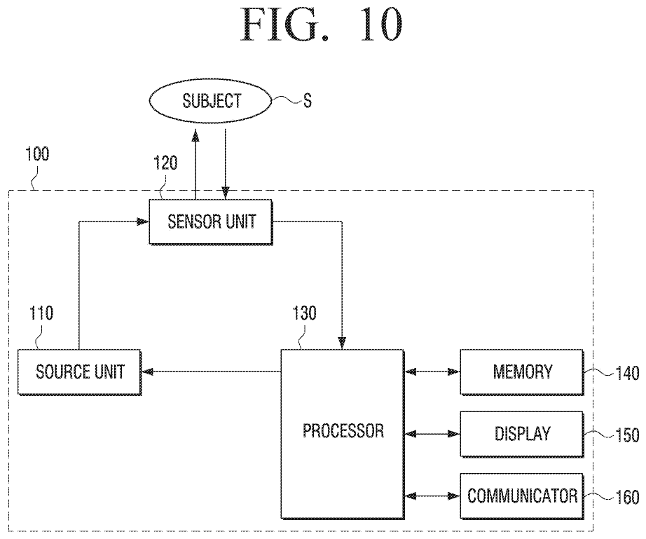

FIG. 10 is a view for describing a configuration of the blood glucose measurement device according to an embodiment of the disclosure, to which the structure of various embodiments described in FIGS. 1 to 9 may be applied, and shows a subject S together with a blood glucose measurement device 100.

Referring to FIG. 10, the blood glucose measurement device 100 may include a source unit 110, a sensor unit 120, a processor 130, a memory 140, a display 150, and a communicator 160. According to the embodiment, some elements may be omitted and suitable hardware/software elements obvious to those skilled in the art may be included in the blood glucose measurement device 100, although those are not shown.

The source unit 110 may generate electromagnetic waves and apply the electromagnetic waves to the sensor unit 120. For example, the source unit 110 may generate microwaves and apply the microwaves to the sensor unit 120. The electromagnetic waves generated by the source unit 110 may pass through the sensor unit 120 and may be input to the processor 130.

The source unit 110 may be implemented as an oscillator and may be implemented as, for example, a voltage-controlled oscillator (VOC).

The sensor unit 120 is an element interacting with the subject S. In particular, the structure of the embodiments described with reference to FIGS. 1 to 9 may be applied to the sensor unit 120, and for example, the sensor unit 120 may include the first resonance sensor 1, the second resonance sensor 2, and the shield layer 4 arranged below the second resonance sensor 2, and may further optionally include the strain sensor 70.

The first resonance sensor 1 and the second resonance sensor 2 may constitute a resonator. The sensor unit 120 may be referred to as a resonator. The resonator may be implemented as a dielectric resonator and there is no limitation to the kinds of dielectrics for implementing the dielectric resonator.

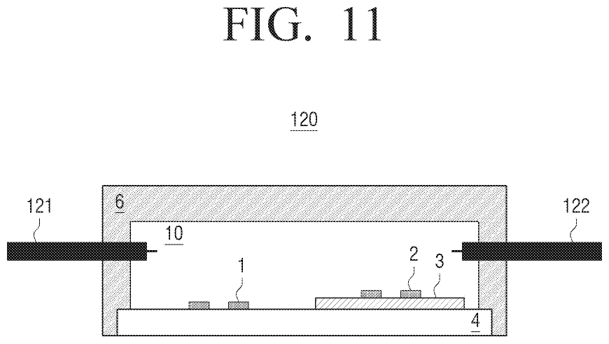

FIG. 11 shows a cross sectional view of the sensor unit 120 according to an embodiment of the disclosure.

Referring to FIG. 11, the sensor unit 120 may include a housing 6 and a space 10 formed of the substrate 4. In this space, the first resonance sensor 1 and the second resonance sensor 2 may be arranged and the shield layer 3 may be arranged below the second resonance sensor 2. A first cable 121 is connected to the source unit 110 to transfer electromagnetic waves to the space 10 and a second cable 122 receives the electromagnetic waves from the space 10. The second cable 122 may be connected to the processor 130.

The processor 130 is an element that is able to controlling general operations of the blood glucose measurement device 100. The processor 130 may include at least one of a CPU, a RAM, a ROM, and a system bus. The processor 130 may be implemented as, for example, a microcomputer (MICOM), application specific integrated circuit (ASIC), or the like.

The processor 130 may measure a power versus frequency with respect to the electromagnetic waves passed through the sensor unit 120.

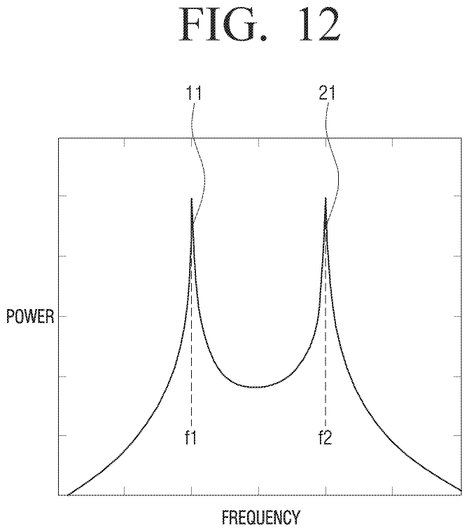

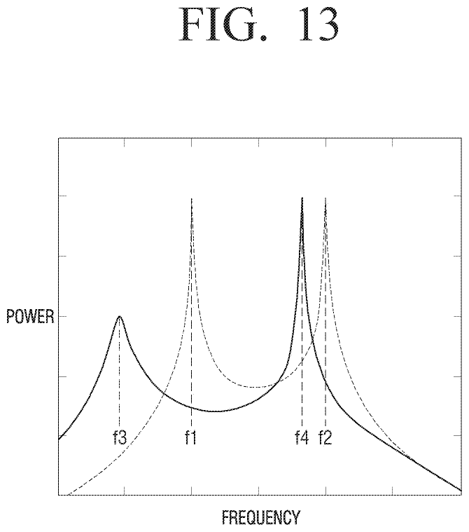

FIG. 12 shows an example of a spectrum of the power versus frequency when there is no subject S. The spectrum of FIG. 12 shows two peaks at frequencies f1 and f2. A peak 11 is obtained by the first resonance sensor 1 and a peak 21 is obtained by the second resonance sensor 2. f1 is a resonance frequency of the first resonance sensor 1 and f1 is a resonance frequency of the second resonance sensor 2. Next, FIG. 13 shows a spectrum when the substrate 4 of the sensor unit 120 reaches or comes into contact with the subject S.

For comparison, FIG. 13 shows the spectrum of FIG. 12 as a dotted line. When the substrate 4 reaches or comes into contact with the subject S, it is confirmed that the resonance frequency of the first resonance sensor 1 has shifted to f3, and the resonance frequency of the second resonance sensor 2 has shifted to f4.

It is confirmed that the shift of the resonance frequency of the second resonance sensor 2 is smaller than the shift of the resonance frequency of the first resonance sensor 1, and this is because the second resonance sensor 2 did not interact with the subject S due to the shield layer 3.

The shift of the resonance frequency of the second resonance sensor 2 is due to the external factors which may affect both the first resonance sensor 1 and the second resonance sensor 2 (for example, temperature or humidity). The shift of the resonance frequency of the first resonance sensor 1 is due to an effect of a blood glucose level of the subject S, in addition to the external factors.

When the subject S reaches or comes into contact with the first resonance sensor 1 and the second resonance sensor 2, the processor 130 may obtain information regarding the blood glucose level of the subject S based on the shift of the resonance frequency of each of the first resonance sensor 1 and the second resonance sensor 2.

Referring to FIGS. 12 and 13, the processor 130 may obtain a difference δ1=f1−f2 between the resonance frequency f1 of the first resonance sensor 1, when there is no subject S, and the resonance frequency f2 of the first resonance sensor 1, when the subject S reaches or comes into contact with the sensor unit 120, and a difference δ2=f3−f4 between the resonance frequency f2 of the second resonance sensor 2, when there is no subject S, and the resonance frequency f4 of the second resonance sensor 2, when the subject S reaches or comes into contact with the sensor unit 120, and the processor 130 may obtain the blood glucose level of the subject S based on δ1 and δ2.

According to an embodiment, the processor 130 may obtain information regarding the blood glucose level corresponding to a value obtained by subtracting δ2 from δ1.

A database regarding the shift of the resonance frequency and the blood glucose level corresponding thereto is constructed in the memory 140, and the processor 130 may determine the blood glucose level corresponding to the value obtained by subtracting δ2 from δ1 from the database, as the blood glucose level of the subject S.

The memory 140 may include, for example, an internal memory or an external memory. The internal memory may include at least one of, for example, a volatile memory (e.g., dynamic RAM (DRAM), static RAM (SRAM), or synchronous dynamic RAM (SDRAM)) and a non-volatile memory (e.g., one time programmable ROM (OTPROM), programmable ROM (PROM), erasable and programmable ROM (EPROM), electrically erasable and programmable ROM (EEPROM), mask ROM, flash ROM, a flash memory (e.g., NAND flash or NOR flash), hard drive, or solid state drive (SSD)).

The external memory may include a flash drive, for example, compact flash (CF), secure digital (SD), micro secure digital (Micro-SD), mini secure digital (Mini-SD), extreme digital (xD), multi-media card (MMC) or a memory stick. The external memory may be functionally and/or physically connected to the blood glucose measurement device 100 via various interfaces.

If the sensor unit 120 further includes the strain sensor 70, the processor 130 may obtain information regarding the blood glucose level of the subject based on δ1, δ2, and sensed data obtained by the strain sensor 70.

The information indicating a corresponding relation between the sensed data obtained by the strain sensor 70 and the shift of the resonance frequency of the first resonance sensor 1 and the second resonance sensor 2 may be stored in the memory 140 in advance. Such information may be provided through machine learning.

The processor 130 may obtain information regarding the blood glucose level of the subject based on the information stored in the memory 140 in advance as described above. For example, based on the information stored in the memory 140 in advance, the processor 130 may confirm the shift δ3 of the resonance frequency of the first frequency sensor 1 due to the shape strain of the first resonance sensor 1 based on the sensed data obtained by the strain sensor 70 for the first resonance sensor 1, and confirm the shift δ4 of the resonance frequency of the second frequency sensor 2 due to the shape strain of the second resonance sensor 2 based on the sensed data obtained by the strain sensor 70 for the second resonance sensor 2. The processor 130 may obtain a value δ5 obtained by subtracting δ3 from δ1 and a value δ6 obtained by subtracting δ4 from δ2, and may obtain information regarding the blood glucose level corresponding to a value obtained by subtracting δ6 to δ5.

In the embodiment, it is described that the noise removal due to shape strain is performed for both the first resonance sensor 1 and the second resonance sensor 2, but the noise removal due to the shape strain may be performed only for the first resonance sensor 1. That is, in such a case the processor 130 may obtain information regarding the blood glucose level corresponding to a value obtained by subtracting δ2 from δ5.

In addition to that the information regarding the blood glucose level may be obtained based on the shift of the resonance frequency, a reflection coefficient, a peak intensity, a bandwidth, and the like may be used to obtain information regarding the blood glucose level. The processor 130 may analyze the shift of the resonance frequency, the peak intensity, the bandwidth, and the like at the same time.

According to an embodiment, the source unit 110 and the processor 130 may be implemented as a vector network analyzer.

The display 150 may include, for example, a liquid crystal display (LCD), a light-emitting diode (LED) display, an organic light-emitting diode (OLED) display (e.g., active-matrix organic light-emitting diode (AMOLED), passive-matrix OLED (PMOLED)), a microelectromechanical systems (MEMS) display, or an electronic paper display.

The processor 130 may control the display 150 to display the information regarding the blood glucose level of the subject S.

According to an embodiment, various guide UIs for blood glucose measurement may be provided through the display 150.

For example, in order to observe the shift of the resonance frequency of the first resonance sensor 1 and the second resonance sensor 2, it is necessary to measure the resonance frequencies of the first resonance sensor 1 and the second resonance sensor 2, when there is no subject S, and accordingly, a UI guiding to bring the subject S into contact with the blood glucose measurement device 100 after a certain period of time may be displayed through the display 150. In another example, a UI explaining which part of the body is to bring into contact with the blood glucose measurement device 100 may be displayed through the display 150.

The communicator 160 is an element for executing communication with an external device. The communicator 160 may be connected to a network, for example, via wireless communication or wired communication to communicate with an external device. The wireless communication may use at least one of, for example, long-term evolution (LTE), LTE Advance (LTE-A), code division multiple access (CDMA), wideband CDMA (WCDMA), universal mobile telecommunications system (UMTS), Wireless Broadband (WiBro), or Global System for Mobile Communications (GSM) as the cellular communication protocol. In addition, the wireless communication may include, for example, near field communication. The near field communication may include at least one of, for example, wireless fidelity direct (WiFi direct), Bluetooth, near field communication (NFC), and Zigbee. The wired communication may include at least one of, for example, universal serial bus (USB), high definition multimedia interface (HDMI), recommended standard 232 (RS-232), or plain old telephone service (POTS). The network may include at least one of communication networks, for example, a computer network (e.g., LAN or WAN), the Internet, or telephone network.

The processor 130 may transmit the information regarding the blood glucose level of the subject S to an external device via the communicator 160.

According to still another embodiment, the blood glucose measurement device 100 may execute the operation of obtaining information regarding the shift of the resonance frequency of the first resonance sensor 1 and the second resonance sensor 2, and the operation of obtaining the information regarding the blood glucose level may be executed in an external device based on this. In such a case, the processor 130 may control the communicator 160 to transmit the shift of the resonance frequency of the first resonance sensor 1 and the second resonance sensor 2 to an external device.

FIG. 10 shows that all of elements are included in the blood glucose measurement device 100, but a blood glucose measurement device according to still another embodiment may not include at least some of elements other than the sensor unit 120. In such a case, the elements which are not included in the blood glucose measurement device may be implemented as external devices of the blood glucose measurement device and such external devices may be connected to the blood glucose measurement device.

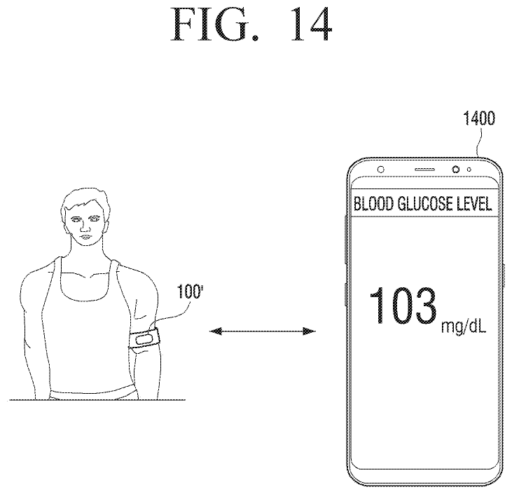

FIG. 14 is a view for describing connection between the blood glucose measurement device and an external device according to an embodiment of the disclosure.

Referring to FIG. 14, a blood glucose measurement device 100′ is connected to an external device 1400 and a blood glucose measurement result may be confirmed in the external device 1400.

For example, the blood glucose measurement device 100′ may transmit the information regarding the shift of the resonance frequency of the first resonance sensor 1 and the second resonance sensor 2 or the information regarding a difference in shift of the resonance frequency of the first resonance sensor 1 and the second resonance sensor 2 to the external device 1400. The blood glucose measurement device 100′ may be connected to the external device 1400 in a communication system such as Bluetooth, Wi-Fi, or NFC. The external device 1400 may obtain the information regarding the blood glucose level based on the information received from the blood glucose measurement device 100′ by using a database provided in itself or a database of an external server, and may display the obtained information regarding the blood glucose level.

According to an embodiment, the external device 1400 may provide various UIs for the blood glucose measurement. For example, various UIs may be provided such as a UI explaining which part of the body is to bring into contact with the blood glucose measurement device 100′, a UI explaining when to bringing the blood glucose measurement device 100′ into contact therewith, a UI for receiving a user input for turning on and off the blood glucose measurement device 100′, and the like.

According to the embodiments described above, it is possible to remove a noise due to the external environment such as a temperature or humidity by using two resonance sensors. In addition, it is necessary to provide the resonance sensors to be spaced apart by a certain distance so that the second resonance sensor 2 is not affected by the subject, and this is suitable to reduce the size of the blood glucose measurement device by using the shield layer 3 having a thickness of several tens micrometers in this disclosure. In addition, it is possible to remove a noise due to the change due to the external force by using the strain sensor.

The embodiments described above may be implemented in a recording medium readable by a computer or a similar device by using software, hardware, or a combination thereof. According to the hardware implementation, the embodiment described in the disclosure may be implemented by using at least one of Application Specific Integrated Circuits (ASICs), digital signal processors (DSPs), digital signal processing devices (DSPDs), programmable logic devices (PLDs), field programmable gate arrays (FPGAs), processors, controllers, micro-controllers, microprocessors, and an electric unit for executing other functions. According to the software implementation, the embodiments such as the procedure and function described in the disclosure may be implemented as separate software modules. Each of the software modules may execute one or more functions and operations described in the disclosure.

The computer instructions for executing the processing operation according to the embodiments of the disclosure described above may be stored in a non-transitory computer-readable medium. When the computer instruction is executed by a processor of a specific device, the computer instructions stored in the non-transitory computer-readable medium may allow the specific device to execute the processing operation of the blood glucose measurement device 100 according to the embodiments described above.

For example, a non-transitory computer-readable medium storing a computer instruction allowing a specific device to execute an operation of obtaining the information regarding the blood glucose level based on the shift of the resonance frequency obtained by the sensor unit 120, when the computer instruction is executed by the processor of the specific device connected to a device including the configuration of the sensor unit 120 described above, may be provided.

The non-transitory computer-readable medium is not a medium storing data for a short period of time such as a register, a cache, or a memory, but means a medium that semi-permanently stores data and is readable by a machine. Specific examples of the non-transitory computer-readable medium may include a CD, a DVD, a hard disk, a Blu-ray disc, a USB, a memory card, and a ROM.

According to an embodiment, the methods according to various embodiments disclosed in this disclosure may be provided to be included in a computer program product. The computer program product may be exchanged between a seller and a purchaser as a commercially available product. The computer program product may be distributed in the form of a machine-readable storage medium (e.g., compact disc read only memory (CD-ROM)) or distributed online through an application store (e.g., PlayStore™). In a case of the on-line distribution, at least a part of the computer program product may be at least temporarily stored or temporarily generated in a storage medium such as a memory of a server of a manufacturer, a server of an application store, or a relay server.

Meanwhile, the blood glucose measurement device has been described hereinabove, but the disclosure may be also used for measurement of not only the blood glucose, but any other substance showing a change in permittivity according to a concentration. That is, the disclosure may be used for measurement of various substances by using a property of changing permittivity according to the concentration thereby changing a resonance frequency of a resonance sensor. For example, the disclosure may be used for measurement of vitamin concentration.

Hereinabove, the preferred embodiments of the disclosure have been shown and described, but the disclosure is not limited to specific embodiments described above, various modifications may be made by those skilled in the art without departing from the gist of the disclosure claimed in the claims, and such modifications may not be individually understood from the technical sprit or the prospect of the disclosure.

What is claimed is:

1. A blood glucose measurement device comprising:
a substrate;
a first resonance sensor and a second resonance sensor arranged on the substrate; and
a shield layer arranged below the second resonance sensor with respect to a direction from the second resonance sensor toward a subject.

2. The device according to claim 1, wherein the first resonance sensor is arranged next to the second resonance sensor in parallel.

3. The device according to claim 1, wherein the first resonance sensor is arranged below the shield layer with respect to the direction from the second resonance sensor toward a subject.

4. The device according to claim 1, further comprising:
an electromagnetic wave transmission layer arranged below the first resonance sensor with respect to a direction from the first resonance sensor toward a subject.

5. The device according to claim 4, wherein the shield layer and the electromagnetic wave transmission layer are formed at the same height.

6. The device according to claim 1, wherein the shield layer is formed to be embedded in the substrate.

7. The device according to claim 1, wherein the shield layer is formed of a ferrite.

8. The device according to claim 1, further comprising:
a processor configured to, based on a subject reaching or coming into contact with the first resonance sensor and the second resonance sensor, obtain information regarding a blood glucose level of the subject based on a shift of a resonance frequency of each of the first resonance sensor and the second resonance sensor.

9. The device according to claim 8, wherein the processor is configured to:
based on a subject reaching or coming into contact with the first resonance sensor and the second resonance sensor, subtract a shift value of the resonance frequency of the second resonance sensor from a shift value of the resonance frequency of the first resonance sensor, and
obtain information regarding a blood glucose level of the subject corresponding to the subtracted value.

10. The device according to claim 1, further comprising:
a communicator comprising circuitry; and
a processor configured to, based on a subject reaching or coming into contact with the first resonance sensor and the second resonance sensor, control the communicator to transmit a shift of a resonance frequency of each of the first resonance sensor and the second resonance sensor to an external device.

11. The device according to claim 1, further comprising:
a strain sensor for detecting a shape strain of the first resonance sensor and the second resonance sensor.

12. The device according to claim 11, wherein the strain sensor comprises a first strain sensor arranged to be adjacent to the first resonance sensor and a second strain sensor arranged to be adjacent to the second resonance sensor.

13. The device according to claim 12, wherein the first strain sensor is arranged to surround the first resonance sensor and the second strain sensor is arranged to surround the second resonance sensor.

14. The device according to claim 11, further comprising:
a processor configured to, based on a subject reaching or coming into contact with the first resonance sensor and the second resonance sensor, obtain information regarding a blood glucose level of the subject based on a shift of a resonance frequency of each of the first resonance sensor and the second resonance sensor and sensed data obtained through the strain sensor.

15. The device according to claim 14, wherein the processor is configured to:
obtain a shift value of a resonance frequency due to shape strain of each of the first resonance sensor and the second resonance sensor based on sensed data obtained by the strain sensor, and,
based on a subject reaching or coming into contact with the first resonance sensor and the second resonance sensor, obtain information regarding a blood glucose level of the subject by subtracting a shift value of the resonance frequency due to the shape strain from a shift value of the resonance frequency of each of the first resonance sensor and the second resonance sensor.
